(19) United States
(12) Patent Application Publication
(10) Pub. No.: US 2005/0107777 A1
(43) Pub. Date: May 19, 2005

(54) Parallel wire ablator

(76) Inventors: Hugh S. West JR., Salt Lake City, UT (US); James B. Logan, Largo, FL (US); Michael J. Decesare, New Port Richey, FL (US)

Correspondence Address: GENE WARZECHA;LINVATEC CORPORATION, 11311 CONCEPT BOULEVARD, LARGO, FL, 33773, US

(21) Appl. No.: 10/712,316

(22) Filed: Nov. 13, 2003

(51) Int. Cl. A61B 18/14

(52) U.S. Cl. 606/41

(57) ABSTRACT

An electrode configuration for use with a standard electrosurgical generator suitable for performing tissue ablation at relatively low power levels. The electrode has at least one curved wire member at its distal tip, the exposed conductive area of the curved wire member being minimized to create high power densities sufficient for tissue ablation. The exposed conductive area of the wire member is partially surrounded by a ceramic insulating support member to enable ablation laterally of the electrode tip as well as proximally and distally. An insulating layer is applied to portions of the outer surfaces of the electrode and the ceramic insulating support member.

### BACKGROUND OF THE INVENTION

1. Field of the Invention

The invention relates to the ablation of tissue during electrosurgical procedures. More particularly, the invention relates to tissue ablation by a monopolar electrosurgical device in a fluid environment during arthroscopic procedures.

2. Description of the Prior Art

Electrosurgical procedures are commonly performed in either a monopolar mode, using a probe having an active electrode placed adjacent tissue to be operated upon, with a return or common electrode placed externally on the patient's body, or a bipolar mode where both active and return electrodes are on the same probe. The procedures utilize radiofrequency (RF) energy to cut or coagulate tissue, these cut and “coag” functions accomplished by applying different energy waveforms and/or power levels to the electrodes. Recently, bipolar electrosurgical devices have been developed for endoscopic issue ablation rather than simply cutting or coagulation. Such new devices require special, dedicated and costly electrosurgical generators, new bipolar electrode designs and high power levels. The term “ablation” in the context of a surgical procedure is generally defined as the removal of tissue by vaporization. Ablation has the connotation of removing a relatively large volume of tissue. Since ablation is the removal of tissue by high-density electrical discharge in a conductive fluid environment, ablation of a sort occurs from the edges of all electrosurgical electrodes used in a cutting mode. This effect, which is independent of whether the return path is provided by a conventional return pad (i.e. monopolar) or a return electrode immersed in the conductive fluid filled space (i.e. bipolar), is related to the volumetric ablation which is the subject of this invention. However, the ablative properties of the invention will be understood to be quite different from known devices.

It is well known to surgeons that for a given electrode design, higher power values give increased rates of tissue removal because the volume of tissue removed (during cutting, for example) is dependent on the power density at the active electrode. This applies to monopolar and bipolar devices. However, until recently, the power density required for tissue ablation has not generally been available over large enough surfaces in known monopolar electrodes. That is why surgeons desiring to perform electrosurgical volumetric ablation often use the aforementioned bipolar ablation systems.

Power density on the surface of a monopolar electrode is somewhat dependent on the conductivity of tissues or fluids in contact with the electrode. The fluids used in electrosurgery are highly conductive and produce non-uniform current density at the electrode surface. Maximizing this power density over large enough surfaces facilitates tissue ablation. The invention facilitates the proper power density over large enough surfaces at power levels lower than the aforementioned bipolar tissue ablation devices.

For an electrosurgical instrument working in a space filled with conductive fluid, such as during an arthroscopic procedure, current density is higher at the edges of the electrode than on its broader or flatter surfaces. When sufficient power is supplied, the current density at the edge of an electrode in this environment is sufficient to raise the temperature of the adjacent fluid thereby making it more conductive. The increased current flow due to this increased conductivity further raises the fluid temperature, which increases the conductivity, which increases the current flow, etc. This continues until the fluid at the electrode edge begins to form a gas phase due to boiling and a luminous discharge becomes visible due to localized arcing. It is believed that the high current density discharge and intense heat at the electrode edge actually perform the ablation. Similarly, bringing the edge of the instrument into contact or sufficiently close proximity with tissue will facilitate initiation of discharge from the edge of the electrode nearest the tissue. If sufficient power is supplied after such high-density discharge is initiated, the instrument can be withdrawn slightly from the tissue while maintaining the high-density discharge at the electrode edge. This phenomenon is well known to surgeons using conventional monopolar electrosurgical instruments.

Although all electrodes used in a cutting mode in a field filled with conductive fluid produce ablation at their edges, not all electrode shapes are equally useful for the removal of relatively large volumes of tissue by ablation. For example, conventional blade-like electrodes are poorly suited for the bulk ablation of tissue due to the small amount of edge area able to produce high density discharge. Similarly, solid cylindrical electrodes also have a small amount of edge area compared to non-edge area. The inherent inefficiency of these shapes necessitates very high power levels relative to the surface area. The efficiency of an electrode for bulk ablation of tissue may be defined as the amount of energy dissipated as high-density ablative discharge divided by the total energy dissipated by the device. Because the electrode is immersed in a conductive fluid, energy will flow from all uninsulated surfaces in contact with the fluid, although energy flowing from non-edge areas will be at a lower density level and will, therefore, dissipate in the fluid with no desirable effect. This low density discharge can be minimized by insulating the non-edge surfaces from the conducting fluid and/or selecting electrode shapes which minimize non-edge surface areas.

The foregoing principles are embodied in a monopolar tissue ablator described in U.S. Pat. No. 6,149,646 (West, Jr. et al.), assigned to the assignee hereof and incorporated by reference herein. The device shown in this patent has a tubular structure that is suitable for large volume tissue ablation in a monopolar mode at relatively low power levels and with conventional electrosurgical generators. This enables electrosurgical ablation with monopolar systems which are simpler and less costly than prior art bipolar devices.

While the device described in the aforementioned U.S. Pat. No. 6,149,646 is effective in many applications, the subject invention relates to a new electrode design which embodies the foregoing principles and expands their utility to new surgical applications. Known prior art ablation electrodes are generally planar structures suitable for the treatment of large, relatively flat tissue surfaces. While these devices may be straight or angled, the working surfaces of the electrodes are generally planar. The working surfaces may be ribbed or otherwise comprise multiple electrodes, but the working surfaces are flat and oriented perpendicularly to the tissue surface to be treated. The invention, however, relates to a curvilinear embodiment of an electrode such that ablation may be performed on tissue surfaces which may be smaller and more curved then those which could be treated with prior art devices.

Known prior art curved electrodes incorporate generally hemispherical tips or other broad curved surfaces which may be used for tissue shrinkage but, because of the inherently low current densities, are not suitable for ablation.

An additional benefit of this invention is that it may be combined with an arthroscopic shaver ablator capable of combining mechanical tissue resection as well as ablation. Such a device is shown in U.S. Pat. Nos. 5,364,395, 5,904,681 and 6,610,059, to West, Jr. all incorporated by reference herein. It would be desirable to have the low power ablation capability of a shaver ablator combining the features of these prior art devices with those of the subject invention.

It is accordingly an object of this invention to produce an electrosurgical tissue ablator suitable for use with conventional electrosurgical generators in a monopolar mode.

It is also an object of this invention to produce a monopolar tissue ablator capable of ablating relatively large volumes of tissue at relatively low power levels.

It is also an object of this invention to produce a monopolar electrode capable of producing high power density levels sufficient for tissue ablation while being driven by relatively low power levels, preferably less than approximately 100 watts in the cut mode and 40 watts in the coag mode.

It is also an object of this invention to produce a monopolar electrode capable of producing tissue ablation within a surgical field filled with conductive fluid.

It is yet another object of this invention to produce an electrode capable of producing tissue ablation along a contoured electrode surface adapted to reach anatomical sites which are not easily accessible with a planar electrode design.

It is an additional object of this invention to produce a monopolar electrode capable of being attached to the curved distal end of an arthroscopic shaver.

### SUMMARY OF THE INVENTION

These and other objects are achieved by the preferred embodiment disclosed herein which is a radiofrequency electrode comprising an elongated shaft having an axis, a proximal end and a distal end; electrical conducting means for conducting radiofrequency energy from the proximal end to the distal end; and at least one electrode member secured to the distal end of the shaft and to the electrical conducting means. The electrode curved is convexly relative to the shaft axis in order to enable retrograde ablation. The electrode member has an outward surface facing away from the axis and an inward surface facing toward the axis, and may be supported by an insulating member interposed between the inward surface and the distal end of the shaft. The insulating member may further include a complementarily shaped channel for supporting the electrode member and for directing ablation effects in a predetermined way. A first portion of the electrode member faces proximally and a second portion of the electrode member faces distally. An aspiration port may be situated adjacent the electrode member and in communication with an aspirating lumen and aspirating means to aspirate ablation by-products through the lumen.

In another aspect, the invention comprises a monopolar electrode for use with an electrosurgical pencil connected to an electrosurgical generator. The electrode comprises a shaft having an axis, a distal end and a proximal end wherein the proximal end is adapted to be connected to the electrosurgical pencil and wherein the distal end terminates in a partially bulbous end having a bulbous electrode supporting surface. A pair of wire-like electrode members is secured relative to the shaft and adapted to substantially conform to the bulbous electrode supporting surface. The electrode members are adapted to receive radiofrequency electromagnetic energy from a source thereof.

### BRIEF DESCRIPTION OF THE DRAWINGS

FIG. 1 is a front perspective view of a monopolar ablator constructed in accordance with the principles of this invention.

FIG. 2 is an enlarged view of the distal end of the instrument shown in FIG. 1.

FIG. 3 is a side elevation view of FIG. 2.

FIG. 4 is a cut away view of FIG. 2.

FIG. 5 is a side elevation view of a portion of the device of FIG. 1 shown during a part of the manufacturing process.

FIG. 6 is a top plan view of the component of FIG. 5 shown assembled with another component during another portion of the manufacturing process.

FIG. 7 is a side elevation view of the component of FIG. 6 shown during yet another portion of the manufacturing process.

FIG. 8 is a cross-section view of FIG. 7 taken along the line 8-8.

FIG. 9 is a side elevation view of the electrode component of the device shown in FIG. 1.

FIG. 10 is a bottom plan view of FIG. 9.

FIG. 11 is a front perspective view of the ceramic insulator component of FIG. 1.

FIG. 12 is a side elevation view of FIG. 11.

FIG. 13 is a top plan view of FIG. 12.

FIG. 14 is a bottom plan view of FIG. 12.

FIG. 15 is a left end view of FIG. 12.

FIG. 16 is a right end view of FIG. 12.

FIG. 17 is a cross-section view of FIG. 13 taken along the line 17-17.

FIG. 18 is an enlarged view of a portion of FIG. 17.

FIG. 19 is a side elevation view of the electrode component of FIG. 9 assembled with the ceramic insulator component of FIG. 12.

FIG. 20 is a top plan view of FIG. 19.

FIG. 21 is a cross-section view of FIG. 20 taken along the line 21-21.

FIG. 22 is a front perspective, partially cut-away, of FIG. 19.

FIGS. 23a-23i are schematic plan views of the distal ends of alternate embodiments of ablator electrodes constructed according to the principles of this invention.

FIG. 24a is a top plan elevation view of another alternate embodiment of an ablator electrode constructed according to the principles of this invention.

FIG. 24b is a side view of FIG. 24a.

FIG. 24c is a top plan elevation view of another alternate embodiment of an ablator electrode constructed according to the principles of this invention.

FIG. 24d is a side view of FIG. 24c.

FIG. 25 is a plan view of a portion of an alternate embodiment of the electrode component shown in FIGS. 9 and 10.

FIG. 26 is a cross-section view of FIG. 25 taken along the line 26-26.

FIG. 27 is front perspective view of an alternate embodiment of FIG. 6.

FIG. 28 is an elevation view of a portion of a shaver ablator constructed in accordance with the principles of this invention.

FIG. 29 is an exploded view of the outer tube of the shaver ablator of FIG. 28 showing the way it would be assembled with an inner tube.

FIG. 30 is an enlarged, inverted view of the distal end of FIG. 29.

### DESCRIPTION OF THE PREFERRED EMBODIMENT

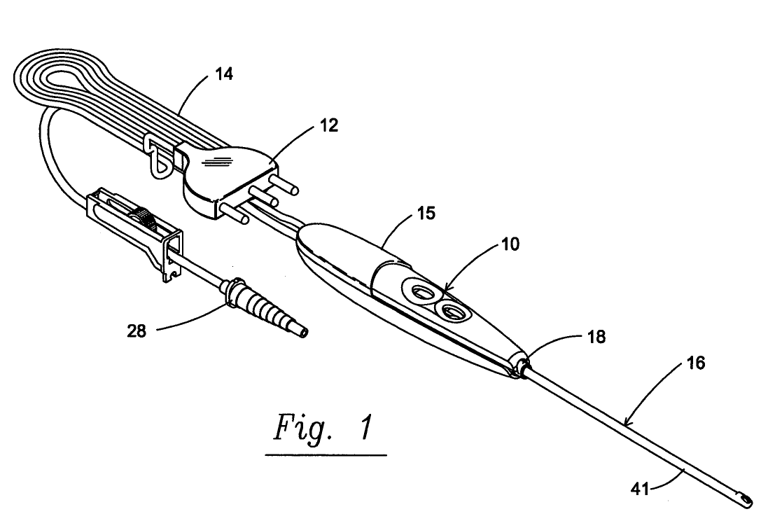

Referring now to FIG. 1, there is shown a monopolar electrosurgical ablator 10 connected to a plug 12 (via a power cord 14) and an aspiration tube 28. Ablator 10 is designed to be plugged into a conventional electrosurgical generator (not shown) and comprises a handle 15 having an ablation electrode 16 extending from its distal end 18, the ablation electrode being constructed in accordance with the principles of this invention.

It will be understood that electrosurgical ablator 10 may comprise an integral, one-piece structure in which the electrode is not separable from the handle, or it may comprise a two-piece structure such as that shown in aforementioned U.S. Pat. No. 6,149,646 in which a separate handle may accept variously sized and shaped electrodes similar to electrode 16 (with or without suction capability).

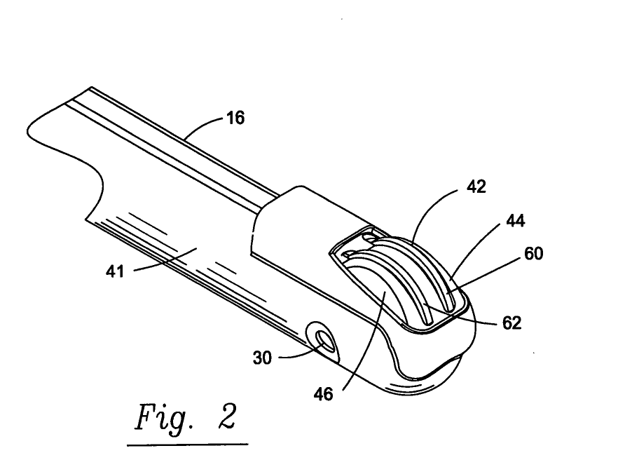

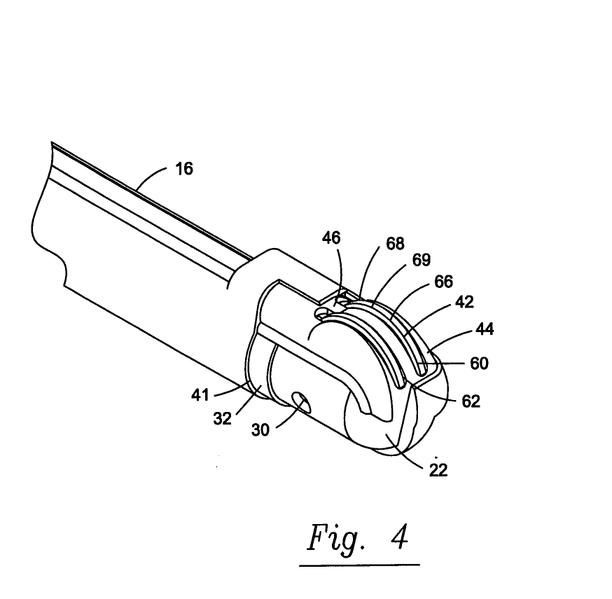

Ablation electrode 16, best seen in FIGS. 2-8 comprises an elongated shaft or tube 22 which is rigid enough to provide firm support for other components described below. Tube 22 is a hollow, electrically conductive cylindrical tube having a radius R, an open proximal end 24, a hemispherical, closed distal end 26 and an axis 27. Distal end 26 is provided with diametrically opposed aspiration ports 30 in communication with the lumen of tube 22 in order to enable the aspiration of ablation by-products (i.e. debris, fluid, bubbles, etc.) from the work site via suction line 28. Alternatively, distal end 26 could be open thereby obviating the need for aspiration ports 30. If ablator 10 is a two-piece structure, tube 22 is secured to and extends from a conventional polymeric hub (not shown) adapted to facilitate connecting the electrode to handle 15. If ablator 10 is a one-piece structure as shown in FIG. 1, tube 22 is fixedly joined to handle 15.

In a preferred embodiment, tube 22 has a diameter of 0.138 inches (3.5 mm) and, if it is electrically conductive, its outer surface is coated with a biocompatible electrical insulating material 32, except for a small proximal area 34 which may be left uncoated to facilitate securing the tube to a hub or to handle 15. A suitable insulating material may be a polymeric shrink-wrap or baked on powder coat ceramic. The insulating coating is preferably uniformly distributed and 0.004-0.007 inches (0.1-0.18 mm) thick. If tube 22 is not electrically conductive, the coating 32 may be omitted.

In a preferred embodiment tube 22 is made of a suitable biocompatible stainless steel. It will be understood, however, that any suitable biocompatible material could be used, even plastic or polymeric material. Furthermore, while tube 22 and ablation electrode 16 are shown to be straight, it will be understood that, with appropriate tube and electrode components, electrode 16 could be bent and/or bendable.

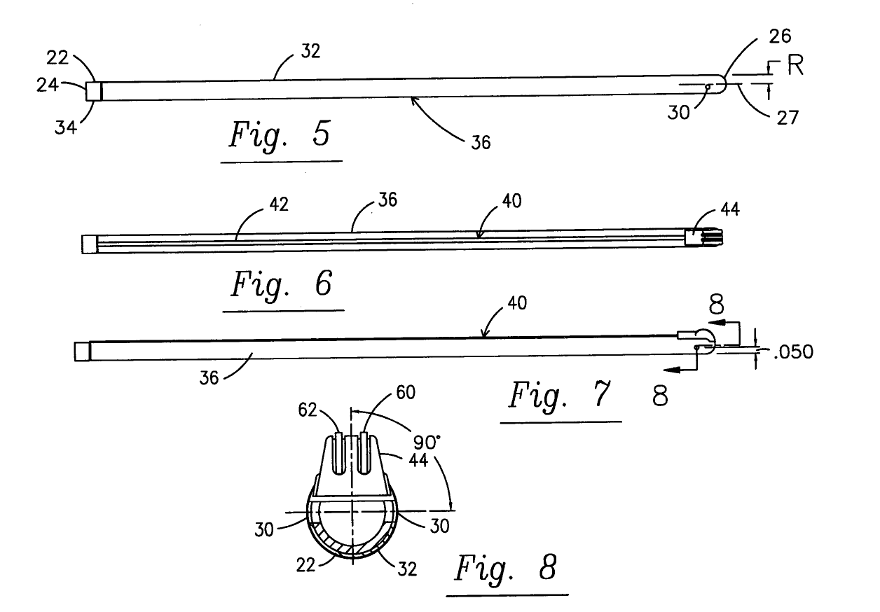

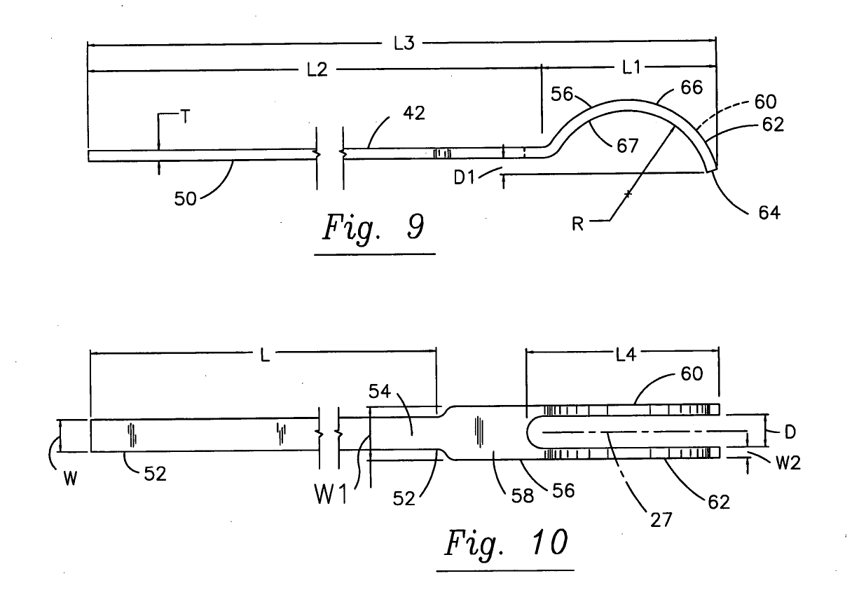

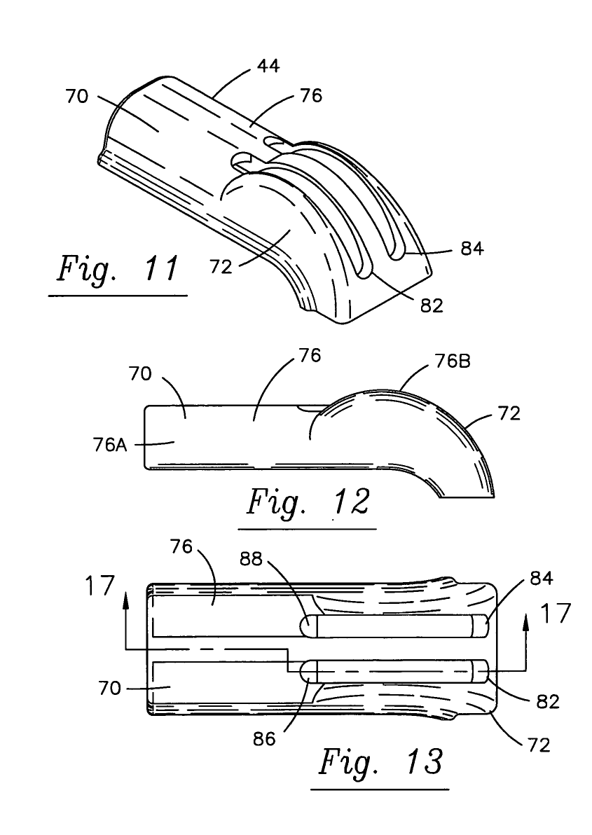

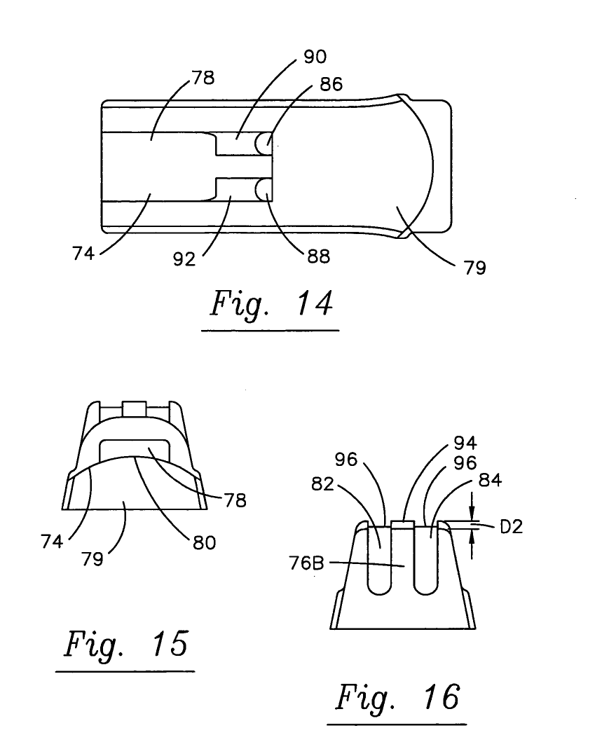

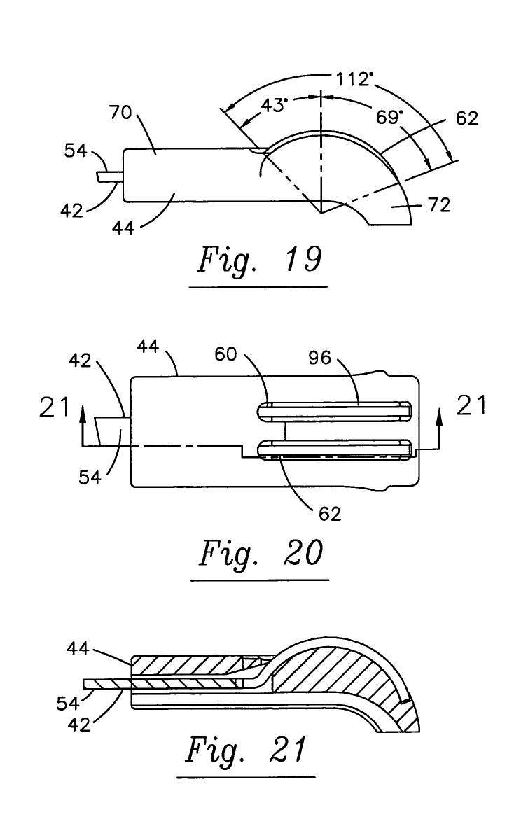

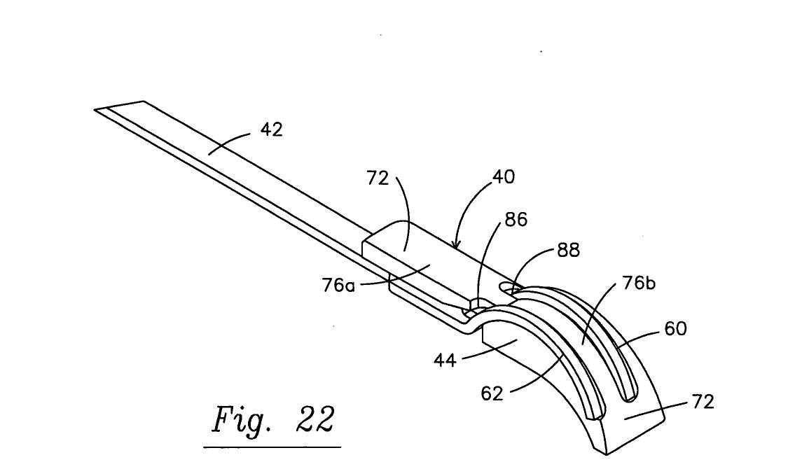

The subassembly of tube 22 with coating 32 is referred to as coated tube 36 which, as best seen in FIGS. 5, 6 and 7, is assembled with electrode subassembly 40. Electrode subassembly 40, best seen in FIG. 22, comprises an elongated, electrically conductive electrode element 42, best seen in FIGS. 9 and 10, joined with ceramic insulator 44, best seen in FIGS. 11-21. The electrode subassembly 40 is adhesively secured to coated tube 36 (using a high temperature medical grade epoxy) at a point displaced 90° from aspiration ports 30. Coated tube 36 and electrode subassembly 40 are then coated with a second layer of insulating material 41 (which may be the same as the first coating layer 32) leaving an exposed electrode window or distal portion 46. This second coating, best seen in FIGS. 2, 3 and 4, is applied to ensure that RF conduction from the ablation electrode 16 to adjacent tissue only occurs at those certain portions of electrode element 42 which will be exposed through distal portion 46, as will be understood below. In the preferred embodiment the thickness of the second coating is uniform and preferably in the range of 0.004-0.007 inches (0.1-0.18 mm). In the preferred embodiment both coatings 32 and 41 are a baked on powder-coating material such as a biocompatible flexible thermoplastic based insulating material having a dielectric strength of at least 800 volts per mil and resistant to temperatures on the order to 300° F.

Electrode element 42 comprises an elongated body portion 50 having a width W, length L and thickness T. Body portion 50 has a proximal end 52 and a distal end 54 which is connected to a pre-formed electrode tip portion 56. Body portion 50 may be curved to conform to the tube or flat. In a preferred embodiment, body portion 50 and tip portion 56 are integrally formed from tungsten as one unitary piece. Tip portion 56 has a base 58 with a width W1 and a pair of convexly curved, parallel wire or wire-like members 60 and 62 extending distally therefrom. Wire members 60 and 62 each have an outwardly facing top surface 66 and an inwardly facing surface 67. Wire members 60 and 62 are spaced apart a distance D and each has a width W2, a length L1 and a degree of curvature adapted to enable electrode tip portion 56 to nest within ceramic insulator 44 as will be understood below. In a preferred embodiment, base portion 52 has a width W equal to 0.030 inches (0.76 mm) while length L is equal to 6.085 inches (154.5 mm), length L1 is equal to 0.165 inches (4.2 mm) and thickness T is equal to 0.010 inches (0.25 mm). The electrode element 42 has the same thickness throughout. Tip portion 56 is curved convexly with the radius of curvature R of the wire members being 0.077 inches (1.96 mm) while length L4 is equal to 0.18 inches (4.6 mm) when L1 equals 0.165 inches (4.2 mm). The distal tips 64 of the wire members 60 and 62 are situated a distance D1 below the bottom surface of body portion 50, where D1 is equal to 0.015 inches (0.38 mm). Each wire member has a width W2 equal to 0.010 inches (0.25 mm) thus providing a wire with a square cross-section and parallel sharp edges 68 and 69 on opposite sides of the top surface 66 of each wire member. The wire members may have cross-sections of varying shapes such as round, oval, elliptical, rectilinear, etc.

In the preferred embodiment each wire member has, as shown in profile in FIG. 9, a single (one dimensional) convex curve within a single plane that is parallel to axis 27. It will be understood that the wire profile could have multiple curves and/or could be compound (three-dimensional).

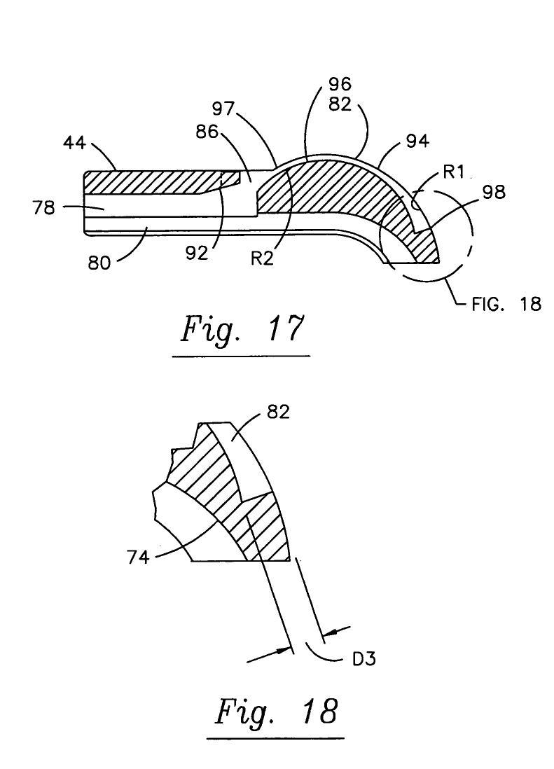

Ceramic insulator 44 is formed to support tip portion 56 and hold wire members 60 and 62 in a particular configuration. While the preferred embodiment of the invention utilizes ceramic insulator 44, as will be understood below the invention may be practiced without such an insulator although the power density at the surface of the wire members would be decreased for any given power level. As used herein, the term “insulator” includes dielectric materials having suitable parameters to enable RF operation of the electrode at selected frequencies. Ceramic insulator 44 has a proximal base portion 70, a distal tip portion 72, an interior surface 74 and an exterior surface 76. Interior surface 74 is adapted to receive electrode base 58 within recess 78. Around recess 78 the interior surface 74 is curved at 80 to conform to the cylindrical outer surface of coated tube subassembly 36. The distal end 79 of interior surface 74 is shaped to conform to the generally hemispherical distal end of coated tube 36. Exterior surface 76 is generally curved transversely at its proximal end 76a (overlying electrode base 58) and longitudinally at its distal end 76b. The distal end of ceramic insulator 44 is provided with a pair of parallel curved channels 82 and 84 adapted to receive wire members 60 and 62, respectively. Apertures 86 and 88 extend through ceramic insulator 44 at the proximal ends of channels 82 and 84, respectively, to enable the wire members to pass therethrough as shown in FIG. 22. Chamfers 90 and 92 form a transition between recess 78 and apertures 86 and 88, respectively. The thickness of the ceramic insulator 44 at all points is variable and is determined in part by the desire to conform the interior surface to the supporting tube and the exterior surface to the shape desired to achieve the intended clinical results. As best seen in FIGS. 16 and 17, each channel 82, 84 lies beneath an adjacent top surface 94 and has a bottom surface 96. The width of each channel is sufficient to receive the width W2 of one of the wire members 60, 62. The radius of curvature R1 of each bottom surface 96 is constant along the length of the channels and is equal to the radius of curvature R of the inwardly facing surfaces 67 of the wire members. The radius of curvature of top surface 94 is continuously variable from a value equal to R1 at proximal channel end 97 to R2 at distal channel end 98. In the preferred embodiment, R1 is 0.077 inches (1.96 mm) and R2 is 0.105 inches (2.67 mm). Both channels 82 and 84 thus have a variable depth D2 along their lengths, terminating in a depth D3 at the distal end of the channels. Depth D2 is less than the thickness T of the wire members over a substantial portion of the channel lengths while D3 is equal to or greater than thickness T. As seen in FIGS. 18-21, this enables the wire members to protrude a desired amount above exterior surface 94 while keeping tissue from inadvertently “catching” on wire ends 64 as electrode 16 is manipulated to and around the worksite.

The projection of the wire members above the exterior surface 94 enables the exposure of a predetermined portion of the conductive area of the electrode surface. The degree of exposure and the power level define the power density at the electrode surface at any given point along the wire member. As mentioned above, the wire member need not have a square cross-section to achieve desirable power densities but could have any cross-sectional shape so as to allow the fabrication of specialized shapes for specific applications. Furthermore, while the channels are complementarily shaped to conform to the shape and size (length, width and thickness) of the wire-like members and to direct ablation in predetermined directions relative to the wire-like members other, non-complementarily shapes could be used to produce different ablation effects.

The small cross-section size of the wire member electrodes enables ablation at low power levels because the ratio of edge area to non-edge area is high. If desired, conduction from selected portions of the wire members can be prevented by changing the shape of the ceramic insulator and/or the channels, thereby altering performance by directing high density discharge to selected areas of the wire members.

In the preferred embodiment electrode element 42 is made of tungsten and is formed from a flat sheet of material. The thickness T of the sheet and the other dimensions of the electrode element may change depending on the desired power levels at which the electrode is intended to operate. In particular, the current density produced by the square cross-section of wire members 60 and 62 projecting from channels 82 and 84 may be varied by changing the relative dimensions disclosed above. Separate wires or wire-like members could be used, with round or other cross-sections.

It is noted that ceramic insulator 44 performs two basic functions. One is to support and insulate the wire members 60 and 62 (from each other and from tube 22 if it is conductive) so that high power densities are achieved along the length of the wire members. Another function is to support the wire members so they maintain their preformed curvilinear profile relative to the distal end of tube 22. Thus, the ceramic insulator has a somewhat bulbous shape on one side of axis 27, thereby enabling the electrode element wire members to face not only laterally relative to axis 27, but proximally and distally. Given suitable insulating dielectric materials, a single support tube having the combined profile of tube 22 and ceramic insulator 44 could be integrally formed of one piece so that a curved electrode could be directly attached (and coated, if necessary), to create window 46.

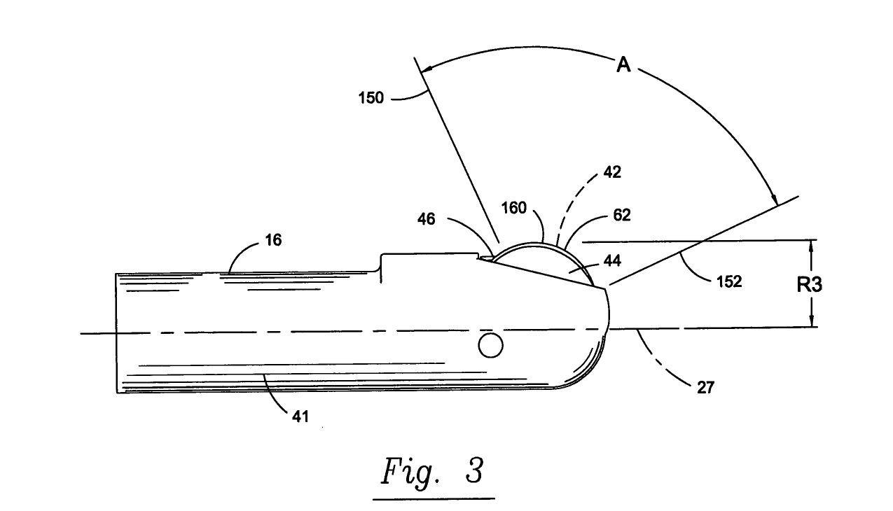

The wire members 60, 62 ablate tissue along their entire exposed length. The ablation occurs primarily on the tissue surfaces tangent to the outer surface 66 of the wire members. Thus, the main area over which ablation may occur can be represented by the arcuate area within which a line may be drawn perpendicular to the tangent points on the exposed wire members. Therefore, as shown in FIG. 3, ablation can occur within area A bounded by proximal boundary line 150 and distal boundary line 152. Lines 150 and 152 are perpendicular to tangents at the proximal-most and distal-most points, respectively, of the portions of wire members 60 and 62 which are not covered by the ceramic insulator 44. The preferred embodiment provides a single electrode design which, without further manipulation, enables ablation laterally, proximally and distally within a plane aligned with the axis of the ablation electrode 16, as well as, areas adjacent to this plane. It will be understood that some ablation effect may be achievable even outside area A, proximal to line 150 and distal to line 152. It will also be understood that area A must be relatively close to wire members 60 and 62 for ablation to occur. The degree of proximity depends upon, among other things, the power level at which ablator 10 is operated, the cross-section profile of the wire members, the ratio of edge to non-edge portions of the wire members, and the type of tissue, etc.

It is noted that the ablative effect of ablator 10 may be achieved laterally relative to axis 27 as well as somewhat distally (closer to line 152) and proximally (closer to line 150). For lateral ablation the wire members have a lateral point 160 that lies along a perpendicular to axis 27 at a radius R3. Points on the wire members distal to point 160 face distally and may effect ablation of tissue distal to the lateral point while points on the wire members proximal to point 160 face proximally and may thereby effect retrograde ablation. This wide range over which ablation may be achieved is made possible with very little increase in the diameter of distal end 26 because of its asymmetrical bulbous design.

Ceramic insulator 44 is a high temperature insulator which serves to electrically insulate all but the exposed wire members of ablator electrode 16 from any conductive fluid or tissue. It must be thick enough and must have a large enough surface area to dissipate enough heat to enable it to continue to insulate the distal end of the electrode without cracking. Any breakage of the ceramic could destroy the ablative action by decreasing power density at the electrode surface below the requisite threshold. Coatings 32 and 41 are preferably sufficiently pliable to enable them to insulate tube 22 and electrode element 42 even if the tube is bent intentionally or unintentionally during use. While the tube is solid stainless steel it may be bent for certain procedures. Also, even if not intentionally bent, sometimes surgeons may inadvertently stress the ablation electrode 16 by using it to push or pry elements during a procedure.

Ceramic material has been chosen for the insulator 44 due to its ability to withstand the high temperatures produced at the electrode distal tip during ablation. It is noted, however, that not all ceramics are able to withstand the high temperatures and thermal gradients present at the distal tip of the electrode. The thermal conductivity and thermal diffusivity of the ceramic have a significant effect on its suitability and performance, more so than absolute strength. The ceramic insulator used in the preferred embodiment is made from an alumina (Al₂O₃) based material AD-998 available from Coorstek of Golden, Colo.

In the preferred embodiment, coatings 32 and 41 are preferably made of high temperature polymeric materials such as, for example, liquid materials which could be used to coat the electrode or granular, particulate materials which could be baked on. The primary requirement is that the material produce a coating having sufficient dielectric strength and suitable flexibility and thermal properties.

While an ablation electrode constructed with the aforementioned dimensions has been found to ablate tissue at input power levels on the order of 20-40 watts in the coag mode and 50-100 watts in the cut mode, it will be understood that satisfactory ablation may occur at various lower and higher power levels with dimensional changes in the electrode. That is, power required is some function of the exposed electrode area.

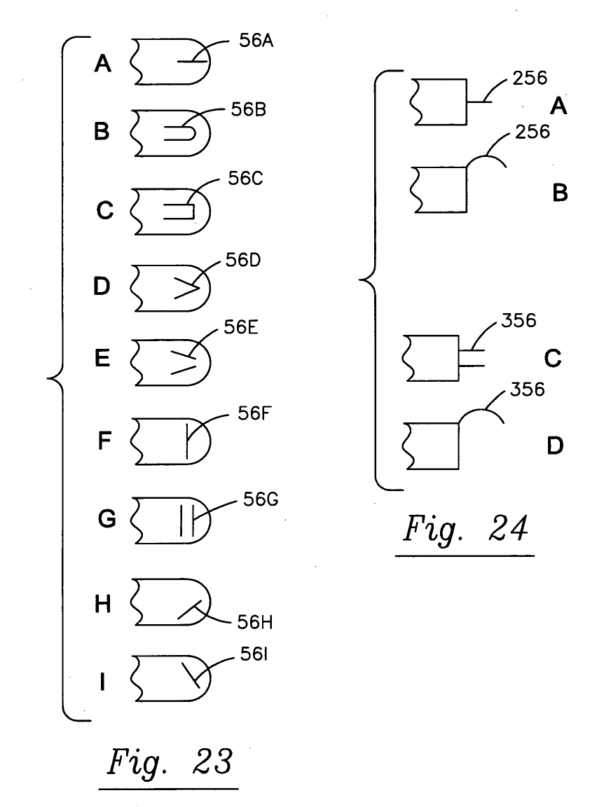

While electrode tip portion 56 is shown here as comprising a pair of parallel wire members, it will be understood that tip portion 56 could comprise one or any number of curved, wire-like members. The term “wire-like” is intended to mean any elongated member having edges or sides suitable for emitting RF energy. The length of the member could be greater than its width, but need not be. As shown in FIGS. 23a-i showing various shapes of wire-like members in plan view at the distal end of a support tube, numerous alternative designs are feasible (with appropriate changes to the associated ceramic insulator used to support the varying shapes.) It is noted that the electrode tip portions 56A-56I may extend longitudinally relative to the axis 27, transversely or in some other direction (partially longitudinally and partially transversely). The side views of each of FIGS. 23a-i are not shown but would have a curvature similar to FIG. 3. FIG. 24a-d shows an alternate embodiment in which electrode ends 256 and 356 extend in a cantilever manner from the end of a support tube without any underlying ceramic or other support. Any of the electrodes of FIG. 23 could be used in the FIG. 24 alternative.

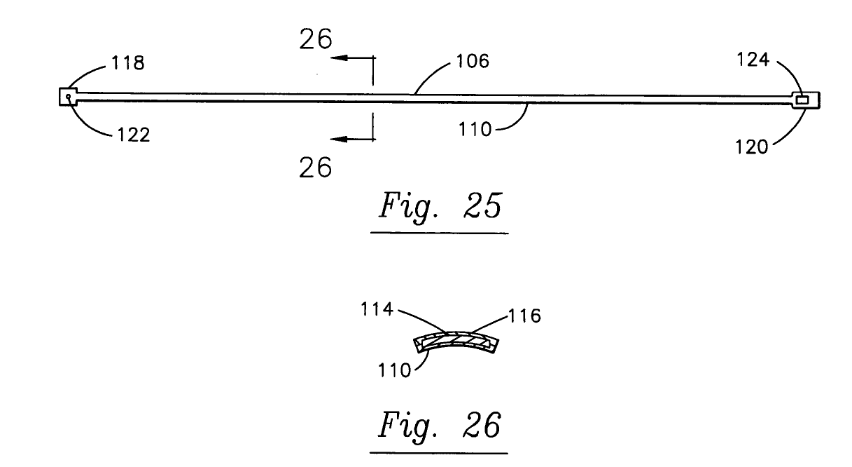

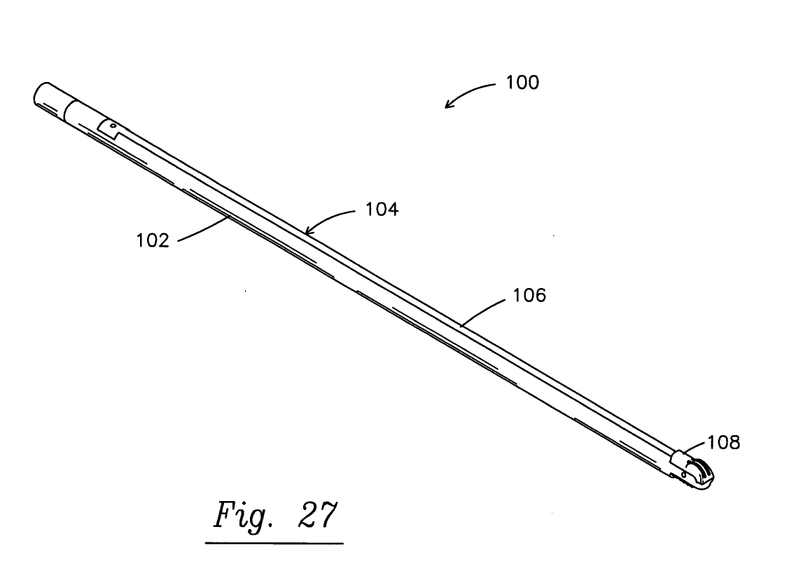

Referring now to FIGS. 25 to 27 there is shown in alternate embodiment of electrode 16. Alternate embodiment 100 (shown uncoated in FIG. 25) comprises metallic or non-metallic tube 102 and electrode subassembly 104. Tube 102 may be identical in all respects to tube 22. Electrode subassembly 104 comprises an elongated electrically conductive electrode element 106 joined with a ceramic insulator 108. Electrode element 106 comprises a printed circuit conductor subassembly 110 (essentially a rigid or flexible printed circuit board) adapted to engage a tip portion (not shown) having the same general structure as tip portion 56. Ceramic insulator 108 may be in all respects identical to ceramic insulator 44.

The primary distinction between electrode 16 and electrode 100 is in the construction of electrode element 106. Electrode element 106 comprises a preformed structure having an elongated printed circuit conductor 110 within a layered structure comprising a bottom insulating layer 112, a middle conducting layer 114 (preferably copper) and a top insulating layer 116. Having both insulating layers may facilitate handling of the electrode subassembly 104. However, whether or not one or both of the insulating layers may be omitted depends on the conductivity of adjacent materials. For example, if tube 102 is non-conductive, the bottom layer may be omitted. If the outer insulating layer is adequate, the top layer may be omitted. The cross-section of printed circuit conductor 110 may be transversely curved as shown in FIG. 26 to conform to the curvature of the tube 102. Printed circuit conductor 110 may be adhesively secured to the tube and has a contact pad 118 at its proximal end and a contact pad 120 at its distal end. Securing printed circuit conductor 110 (and, therefore, electrode subassembly 104) to the tube may facilitate manufacture, but may be unnecessary if the outer insulating coating is adapted to hold the parts together. Contact pads 118 and 120 are junction points which include extensions of conductive layer 112 which, in the preferred embodiment, are covered by insulating layers 112 and 116. Contact pad 118 is provided with an aperture 122 or other means to enable a solder or other connection to other components supplying radiofrequency energy to the conductive layer of electrode element 106. Contact pad 120 is provided with an aperture 124 to enable a solder or other connection to a tip portion similar to tip portion 56, but adapted to engage the conductive layer via aperture 124.

It will be understood that electrode 100 requires only a single outer coating of insulating material comparable to the second coating 41 provided on electrode 16. That is, the need for the first coating is eliminated due to the construction of the electrode element 106, whether or not tube 22 is conductive. This structure also lends itself not only to a smaller diameter ablation electrode 16 (because of the elimination of one coating layer) but also to bendable electrodes if printed circuit subassembly 110 is made flexible so it can be bent along with tube 102.

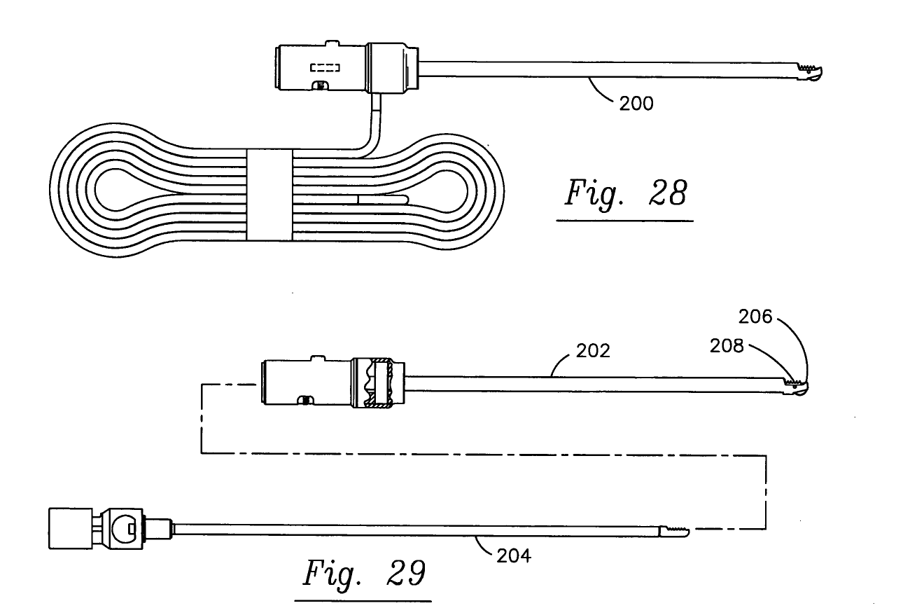

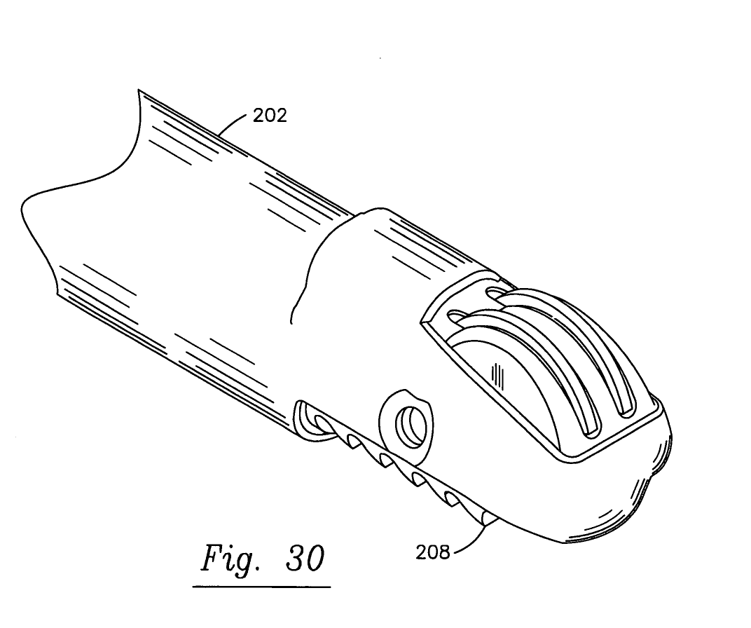

Referring now to FIGS. 28 through 30, there is shown an alternate embodiment of the invention incorporated in a shaver ablator 200. Shaver ablator 200 is in part a mechanical resection instrument comprising an outer tube 202 and an inner tube 204 rotatable relative to outer tube 202 in a conventional manner. Outer tube 202 and inner tube 204 each comprise a cutting window at their distal ends. Tissue is resected as the inner cutting window moves, in this case rotates, past the outer cutting window. The construction of outer tube 202 is similar to the construction of either electrode 16 or 100, with the only difference being that the tubular body of outer tube 202 is comparable to the body of electrodes 16 and 100 and is adapted via opening 206 and/or teeth 208 to operate as a mechanical arthroscopic shaver.

The preferred embodiment of ablation electrode 16 incorporates a bulbous and asymmetrical insulator and curved wire-like members. As used herein, the term “bulbous” means that a portion of the electrode surface such as lateral point 160 faces laterally and is spaced from axis 27 a distance R3 greater than the radius of the support tube after it has been coated, and another portion faces proximally. It will be understood that the bulbous profile could be symmetrical with, for example, another insulator/wire-like member subassembly situated diametrically opposite to the first. The second subassembly could be situated at some angle other than 180° relative to the first.

While the preferred embodiment is a monopolar system, it will be understood that a bipolar configuration could be produced by incorporating a return electrode in proximity to the distal tip portion 56 of electrode element 42.

It will be understood by those skilled in the art that numerous improvements and modifications may be made to the preferred embodiment of the invention disclosed herein without departing from the spirit and scope thereof.

1. A radiofrequency electrode comprising:
an elongated shaft having an axis, a proximal end and a distal end;
electrical conducting means for conducting radiofrequency energy from said proximal end to said distal end; and
at least one electrode member secured relative to said distal end of said shaft and to said electrical conducting means, said at least one electrode curved convexly relative to said shaft axis.

2. A radiofrequency electrode according to claim 1 wherein said electrode member has an outward surface facing away from said axis and an inward surface facing toward said axis, further comprising an insulating member interposed between said inward surface and said distal end of said shaft.

3. A radiofrequency electrode according to claim 2 wherein said insulating member comprises a channel for supporting said electrode member.

4. A radiofrequency electrode according to claim 1 wherein said electrode member is elongated and lies in a plane.

5. A radiofrequency electrode according to claim 4 wherein said plane is axial.

6. A radiofrequency electrode according to claim 4 wherein said plane is parallel to said axis.

7. A radiofrequency electrode according to claim 4 wherein said plane is transverse to said axis.

8. A radiofrequency electrode according to claim 1 wherein said electrode member is a wire-like member.

9. A radiofrequency electrode according to claim 1 wherein said electrode member is curved in one dimension only.

10. A radiofrequency electrode according to claim 1 wherein a first portion of said electrode member faces proximally and a second portion of said electrode member faces distally.

11. A radiofrequency electrode according to claim 10 wherein the surface of said distal end of said shaft is spaced a first predetermined distance from said axis and wherein at least a part of said first portion of said electrode member is spaced a second predetermined distance from said axis, said second predetermined distance being greater than said first predetermined distance.

12. A radiofrequency electrode according to claim 1 further comprising:
a longitudinally extending lumen;
an aspiration port situated adjacent said electrode member and in communication with said lumen; and
aspirating means to aspirate ablation by-products through said lumen.

13. A radiofrequency electrode according to claim 12 wherein said lumen is within said shaft.

14. A radiofrequency electrode according to claim 1 further comprising a mechanical resection instrument comprising relatively moving inner and outer cutting windows.

15. An ablation device comprising:
a handle;
an elongated shaft extending from said handle, said shaft having a proximal end attached to said handle and a distal end, said distal end terminating in a generally cylindrical closed end;
an electrode supporting member secured to said distal end, said electrode supporting member having an inner surface for conforming to said distal end and an outer bulbous surface for supporting at least one electrode; and
at least one electrode member secured relative to and conforming to said bulbous electrode supporting surface, said electrode member having a proximal end and a distal end and adapted to receive radiofrequency electromagnetic energy from a source thereof.

16. An ablation device according to claim 15 wherein said electrode member is a wire-like member and further comprising two said wire-like members.

17. An ablation device according to claim 16 wherein said electrode members are parallel.

18. An ablation device according to claim 17 wherein said electrode members are axially aligned.

19. An ablation device according to claim 15 wherein said bulbous electrode supporting surface is electrically non-conductive.

20. An ablation device according to claim 17 wherein said electrode members each have a conducting surface parallel to and spaced a predetermined amount away from said insulating surface.

21. An ablation device according to claim 15 wherein said ablation device operates in a liquid medium and further comprising:
an aspiration means for aspirating ablation by-products from said liquid medium, said aspiration means comprising:
at least one distal port situated at said distal end of said shaft;
a longitudinally extending lumen on said shaft, said lumen operatively connected to said distal port; and
means for aspirating ablation by-products through said distal port and said lumen.

22. An ablation device according to claim 15 wherein said electrode member is a monopolar electrode.

23. An ablation device according to claim 15 further comprising a return electrode adjacent said distal end whereby said ablation device is a bipolar device.

24. An ablation device according to claim 16 wherein the distal-most ends of said electrode members are electrically connected by a transverse conductor.

25. An ablation device according to claim 15 wherein said distal end of said elongated shaft has an outer cylindrical surface spaced a first predetermined radial distance from said axis, and wherein said bulbous electrode supporting surface has a portion thereof at a second predetermined radial distance from said axis, said second predetermined distance being greater than said first predetermined distance.

26. An ablation device according to claim 25 wherein said distal end has a first predetermined width in a first plane and a second predetermined width in a second plane perpendicular to said first plane, said first predetermined width being greater than said second predetermined width.

27. An ablation device according to claim 26 further comprising at least one aspirating port in the portion of said distal end having said first predetermined width.

28. An ablation device according to claim 27 further comprising an aspirating port on opposed sides of said portion of said distal end.

29. A monopolar electrode for use with an electrosurgical pencil connected to an electrosurgical generator comprising:
a shaft having an axis, a distal end and a proximal end, said proximal end adapted to be connected to said electrosurgical pencil, said distal end terminating in a partially bulbous end having a bulbous electrode supporting surface; and
a pair of wire-like electrode members substantially conforming to said bulbous electrode supporting surface, said electrode members adapted to receive radiofrequency electromagnetic energy from a source thereof.

30. A radiofrequency electrode according to claim 1 wherein said electrical conducting means comprises a preformed printed circuit board subassembly.

31. A radiofrequency electrode according to claim 30 further comprising securing means to secure said printed circuit board subassembly to said elongated shaft.

32. A radiofrequency electrode according to claim 30 wherein said printed circuit board subassembly is sufficiently flexible to enable said elongated shaft to be bent without breaking said electrical conducting means.

33. A radiofrequency electrode according to claim 30 further comprising a first junction means to connect said electrical conducting means to a source of radiofrequency energy and a second junction means to connect same to said at least one electrode member.
